(19) United States
(12) Patent Application Publication
(10) Pub. No.: US 2005/0021012 A1
(43) Pub. Date: Jan. 27, 2005

(54) Laser treatment for reducing wrinkles

(76) Inventor: Eric F Bernstein, Gladwyne, PA (US)

Correspondence Address: LICATLA & TYRRELL P.C., 66 E. MAIN STREET, MARLTON, NJ, 08053, US

(21) Appl. No.: 10/497,812

(22) PCT Filed: Dec. 12, 2002
(86) PCT No.: PCT/US02/39718

Related U.S. Application Data
(60) Provisional application No. 60/340,208, filed on Dec. 12, 2001.

(51) Int. Cl. A61B 18/18

(52) U.S. Cl. 606/9

(57) ABSTRACT

Methods for diminishing or reducing pore and hair follicle size in a subject and decreasing or inhibiting undesired activities of enlarged pores such as oily skin, acne and rosacea are provided wherein the skin of a subject is irradiated, preferably via a laser, with electromagnetic radiation at a wavelength sufficient to irradiate vessels surrounding cutaneous sebaceous glands located in the middermis so that an inflammatory response against cutaneous sebaceous glands and surrounding stroma is stimulated.

This application claims the benefit of priority from U.S. Provisional Application No. 60/340,208, filed Dec. 12, 2001, which is herein incorporated by reference in its entirety.

### BACKGROUND OF THE INVENTION

Enlarged pores and/or hair follicles are associated with sun-damage and acne/rosacea as well as additional factors such as heredity. This condition can pose a significant clinical problem.

Enlarged pores and/or hair follicles are treated primarily with topical agents such as retinoids and alpha-hydroxy acids. Improvement from these agents is achieved primarily by the ability of these agents to remove debris contained within the pore and/or follicles. These topical agents typically act slowly producing only moderate improvement.

Accordingly, there is a need for better treatments to diminish or reduce pore and or hair follicle size.

### SUMMARY OF THE INVENTION

An object of the present invention is to a provide a method for diminishing or reducing pore and/or hair follicle size in a subject which comprises irradiating skin of a subject with electromagnetic radiation at a wavelength sufficient to irradiate vessels surrounding cutaneous sebaceous glands located in the mid-dermis so that an inflammatory response against cutaneous sebaceous glands and surrounding stroma is stimulated and pore and/or hair follicle size is reduced or diminished. In a preferred embodiment, a laser is used to administer the radiation to the skin of the subject. A pulsed, scanned, or continuous laser light in the range of approximately 580 to 700 nanometers is preferred. This method for decreasing pore and/or hair follicle size method may further comprise administration of a topical agent such as a retinoid, alpha-hydroxy acid, or antioxidant, microdermabrasion or chemical peels for the purpose of reducing enlarged pores.

Another object of the present invention is to provide a method for reducing and/or inhibiting undesired activities of enlarged pores including, but not limited to oily skin and acne or rosacea which comprises irradiating skin of a subject with electromagnetic radiation at a wavelength sufficient to irradiate vessels surrounding cutaneous sebaceous glands located in the mid-dermis so that an inflammatory response against cutaneous sebaceous glands and surrounding stroma is stimulated and pore and/or hair follicle size is reduced or diminished. In this method it is also preferred that a laser be used to administer the radiation to the skin of the subject. A pulsed, scanned, or continuous laser light in the range of approximately 580 to 700 nanometers is preferred. This method for reducing and/or inhibiting undesired activities of enlarged pores may further comprise administration of a topical agent such as a retinoid, alpha-hydroxy acid, or antioxidant, microdermabrasion or chemical peels for the purpose of reducing undesired activities of enlarged pores.

### DETAILED DESCRIPTION OF THE INVENTION

The present invention provides methods for reducing, diminishing or decreasing pore and/or hair follicle size in the skin of a subject as well as methods for reducing or inhibiting undesired activities of enlarged pores and/or hair follicles including, but not limited to oily skin, acne and rosacea. These methods are particularly useful in reducing the size of enlarged pores secondary to cutaneous photodamage.

In the methods of the present invention, the skin of a subject is irradiated with light (electromagnetic radiation) at a wavelength sufficient long to enable penetration of light to the sebaceous glands. The light needs to be of a wavelength that penetrates through an entire vessel and does not deposit energy superficially on the surface of a vessel. Accordingly, light in the range of approximately 580 and 700 nanometers capable of irradiating the vessels surrounding the cutaneous sebaceous glands that are located in the mid-dermis is preferred. Light of this wavelength will stimulate an inflammatory response directed against the vessels in the vicinity of the sebaceous glands, and against the sebaceous glands themselves. The resulting inflammatory response helps diminish the size of the glands as well as remodels the tissue surrounding the glands thereby resulting in a reduction in pore and/or hair follicle size and a decrease in oily skin and acne.

In a preferred embodiment of the method, a laser is used to administer the radiating light. The laser preferably administers light at a wavelength between approximately 580 and 700 nanometers. The light from the laser can be administered as a pulse, scan or a continuous beam that is swept across the skin giving the effect of a pulse. Scan speed (or the dwell time of a beam in a particular area) is preferably selected to be consistent with the beam-size and power of a continuous beam so that the dwell time of radiation at a point being treated (due to the time taken for a beam of finite size to pass that point) delivers the appropriate fluence. An example of a suitable laser for providing radiation in the inventive treatment of enlarged pores is a frequency doubled Nd:YAG laser. However, as will be understood by those of skill in the art upon reading this disclosure, other lasers that deliver electromagnetic light in the wavelength range of approximately 580-700 nm can be used.

Laser treatment of the facial skin of ten subjects in this wavelength range resulted in a decrease in pore size of approximately 40%. Further, half of the subjects reported a noticeable decrease in oily skin and acne.

The laser treatment method of the present invention may be used alone or in combination with other treatments used for the purpose of reducing enlarged pores and/or undesired activities of enlarged pores. Examples of such treatments, which can be used in combination with the laser treatment of the present invention, include, but are not limited to, administration of topical agents such as alpha-hydroxy acids, retinoids, and antioxidants, microdermabrasion and chemical peels.

The following nonlimiting example is provided to further illustrate the present invention.

### EXAMPLE

### Example 1

### Treatment of Subjects

Experimentally, treatments were administered to the skin of the face of subjects using a pulsed-dye laser. Electromagnetic radiation (light) was administered using a 595 nm light, a 10 mm spot, a pulse duration of 1.5 milliseconds, and an average fluence of 5.5 Joules per square centimeter. Ten subjects were treated an average of three times. Subjects estimated an average reduction in pore size of 40%. Fifty percent of subjects also reported a noticeable decrease in oily skin and acne.

### REFERENCES

Anderson R R, Parrish J A. Microvasculature can be selectively damaged using dye lasers: a basic theory and experimental evidence in human skin. Lasers Surg Med 1981; 1(3):263-76. Parrish J A, Anderson R R, Harrist T, Paul B, Murphy G F. Selective thermal effects with pulsed irradiation from lasers: from organ to organelle. J Invest Dermatol 1983;80 Suppl:75s-80s.
Anderson R R, Jaenicke K F, Parrish J A. Mechanisms of selective vascular changes caused by dye lasers. Lasers Surg Med 1983;3(3):211-5.
Anderson R R, Parrish J A. Selective photothermolysis: precise microsurgery by selective absorption of pulsed radiation. Science 1983;220(4596):524-7. Bernstein E F, Lee J, Lowery J, Brown D B, Geronemus R, Lask G, et al. Treatment of spider veins with the 595 nm pulsed-dye laser [see comments]. J Am Acad Dermatol 1998;39(5 Pt 1):746-50.
Zelickson B D, Kilmer S L, Bernstein E, Chotzen V A, Dock J, Mehregan D, et al. Pulsed dye laser therapy for sun damaged skin. Lasers Surg Med 1999;25(3):229-36.

1. A method for diminishing or reducing pore and or hair follicle size in a subject comprising irradiating skin of a subject with electromagnetic radiation at a wavelength sufficient to irradiate vessels surrounding cutaneous sebaceous glands located in the mid-dermis so that an inflammatory response against cutaneous sebaceous glands and surrounding stroma is stimulated and pore and hair follicle size is reduced or diminished.

2. The method of claim 1 wherein the skin is irradiated with a pulsed, scanned, or continuous laser light in a wavelength range of 580 to 700 nanometers.

3. The method of claim 1 wherein the enlarged pores are secondary to cutaneous photodamage.

4. The method of claim 1 further comprising topical administration of an alpha-hydroxy acid, a retinoid, or an antioxidant, microdermabrasion or administration of a chemical peel that reduces pore and hair follicle size.

5. A method for reducing or inhibiting undesired activities of enlarged pores or hair follicles in a subject comprising irradiating skin of a subject with electromagnetic radiation at a wavelength sufficient to irradiate vessels surrounding cutaneous sebaceous glands located in the mid-dermis so that an inflammatory response against cutaneous sebaceous glands and surrounding stroma is stimulated and undesired activities of enlarged pores and hair follicle size is reduced.

6. The method of claim 5 wherein the undesired activities cause oily skin, acne or rosacea.

7. The method of claim 5 wherein the skin is irradiated with a pulsed, scanned, or continuous laser light in a wavelength range of 580 to 700 nanometers.

8. The method of claim 5 further comprising topical administration of an alpha-hydroxy acid, a retinoid, or an antioxidant, microdermabrasion or administration of a chemical peel that reduces pore and hair follicle size.
